(19) United States
(12) Patent Application Publication
(10) Pub. No.: US 2003/0208223 A1
(43) Pub. Date: Nov. 6, 2003

(54) Device incorporating a hollow element for being positioned along a body cavity of a patient and method of positioning the same

(76) Inventor: Daniel Eduard Kleiner, Newcastle (AU)

Correspondence Address: Ropes & Gray, One International Place, Boston, MA, 02110-2624, US

(21) Appl. No.: 10/239,590

(22) PCT Filed: Mar. 22, 2001
(86) PCT No.: PCT/IB01/00444

(30) Foreign Application Priority Data
Mar. 23, 2000 (AU) PQ 6414

(51) Int. Cl. A61M 29/00

(52) U.S. Cl. 606/198

(57) ABSTRACT

There is provided apparatus (14) incorporating an elongate hollow element (22) for being positioned along a body cavity of a patient. The hollow element has a leading region and a trailing region and is arranged for being progressively everted along the hollow element from the leading region to thereby be increasingly extended for progressively lining the body cavity as the trailing region follows along. The apparatus generally further comprises an enclosure comprising an insertion head (16) for being inserted into an entrance of the body cavity and a flexible bag (18) in which the hollow element is housed. The hollow element is extended from the insertion head by increasing pressure within the enclosure. Methods for positioning the hollow element along the body are disclosed. The apparatus is particularly suitable for use as a tamponade device for use in the lower gastrointestinal tract for stemming internal bleeding.

### FIELD OF THE INVENTION

The present invention relates to a device having use in facilitating examination or treatment within a body cavity of a patient as well as a method relating to the use of the device. Various different embodiments are provided for enabling treatments or examination within the body cavity to be performed in different ways.

### BACKGROUND OF THE INVENTION

Tamponade devices incorporating balloons for applying pressure to bleed sites in the gastrointestinal tract for instance upon inflation of the balloon are known in the art. To assist in halting the bleeding, the balloon may be coated with anticoagulants or other substances for being applied to the bleed site. Devices of this type are disclosed in for instance U.S. Pat. Nos. 5,709,657 and 5,906,587.

However, in order to deploy such devices it is necessary to know the location of the bleed site within a relatively limited distance range. This may require that the patient be examined endoscopically or by other means which results in a delay in the patient receiving treatment. This is highly undesirable in emergency situations where it is critical for the well being of the patient that treatment be administered rapidly.

In addition, an instrument like an endoscope requires that the attendant be sufficiently trained and skilled in its use. In serious bleeds, locating the bleed site with an endoscope can be difficult and time consuming, even when the attendant is skilled in the use of the instrument.

A catheter incorporating an everted balloon arrangement is disclosed patent application EP 0227583 for dilating occluded blood vessels. In particular, the balloon is arranged within a housing for being expelled therefrom by a telescopic sheath slidable longitudinally along the catheter to force the balloon from the catheter in conjunction with the application of fluid pressure to the interior of the balloon. The balloon is everted as it is forced from the catheter and is guided along the interior of the blood vessel by the sheath. When in position, the balloon is inflated to thereby dilate the occlusion.

Further catheters of this general type are disclosed in patent applications WO 96/22122 and DT 2406823, and in U.S. Pat. No. 5,328,469.

In U.S. Pat. No. 4,271,839 a further catheter for use in dilating occluded blood vessels is disclosed. In this device, an elastic balloon is arranged in an inverted condition in a distal end of the catheter. In use, the catheter is located in close proximity to the occlusion and the balloon everted from the catheter by fluid pressure applied to the interior of the balloon. The balloon is extruded from the catheter in anisotropic fashion in advance of substantial lateral expansion. Upon the balloon being fully extended, the balloon is inflated and thereby expanded laterally into contact with the occlusion to thereby line the surrounding blood vessel wall for effecting dilation of the occlusion. To retract the balloon the fluid pressure within the balloon is lowered and a cord attached to the interior of the free end of the balloon is then retracted causing the balloon to be withdrawn into the catheter.

In International Patent Application No. WO 87/05523 there is disclosed a tubular device incorporating an flexible element arranged in an invaginated conformation for being located along a body orifice or duct. The device incorporates an outer tubular support housing the flexible element and a stiff push tube slidable within the outer support for causing the element to be progressively everted as it is forced from the outer support by the push tube. The push tube, therefore, not only drives the everting of the element but also guides the element along the relevant duct or body orifice as the flexible element is being extended from the outer support.

A further such device described in WO 87/05523 for sealing the nasal fossae comprises a sealed tube in which a balloon is housed in one end thereof and a plunger is received in an opposite end. Upon progression of the plunger along the tube the balloon is caused to be everted and fully extended from the tube. With further progression of the plunger, the balloon is inflated radially to thereby be brought into contact with the surrounding wall defining the body cavity or duct and so fill the surrounding volume. Such devices as disclosed in WO 87/05523 are, therefore, only suitable for following substantially straight paths of progression within the body or in the later type of device, for filling a surrounding void where a bleed site is known to exist.

However, as indicated above, in many situations the site of a bleed within, for instance, the lower gastrointestinal tract is unknown and indeed, the path to the bleed site may be sinuous or deviate significantly from a straight path. Accordingly, it is desirable that methods and apparatus be provided which may be used in such applications.

### SUMMARY OF THE INVENTION

It is an aim of the present invention to ameliorate one or more problems of the prior art or to at least provide an alternative to the prior art.

Broadly, the invention is based on the recognition that eversion of a hollow element from an invaginated conformation to an extended conformation may be utilised to achieve positioning of the hollow element along a body cavity of a patient.

Accordingly, in one aspect of the invention there is provided a method of positioning an elongate hollow element along a body cavity in a patient, wherein the hollow element has a leading region and a trailing region, and the method comprises:

causing the hollow element to be everted progressively along the hollow element from the leading region thereof such that the hollow element is forced to be increasingly extended and thereby progressively line a surrounding wall defining the body cavity as the trailing region follows along; and

wherein the hollow element self navigates along within the body cavity following a path determined by the surrounding wall defining the body cavity.

Preferably, the hollow element will be inflatable for causing the hollow element to be progressively everted.

Typically, the hollow element will be adapted, for limiting swelling during the inflation thereof thereby preferentially causing the trailing region to be drawn along.

Hence, in another aspect of the present invention there is provided a method of positioning an inflatable hollow element in a body cavity of a patient, wherein the hollow element has a leading region and a trailing region, and the method comprises:

inflating the hollow element to cause the hollow element to be everted progressively therealong from the leading region and thereby be increasingly extended into the body cavity as the trailing region of the hollow element follows along; and

wherein the hollow element is adapted for limiting swelling during the inflation thereof thereby preferentially causing the trailing region to be drawn along.

To assist positioning the hollow element along the body cavity and in particular, to assist travel of the hollow element around a bend or sinuous path, the hollow element may be vibrated. This may be achieved by coupling a vibration source to apparatus incorporating the hollow element such that when operated, the vibrations from the vibration source are transmitted along the hollow element.

In yet another aspect of the invention there is provided a device incorporating an inflatable hollow element for being positioned along a body cavity of a patient, wherein the hollow element has a leading region and a trailing region and is arranged for being everted progressively along the hollow element from the leading region thereof to thereby be increasingly extended for progressively lining a surrounding wall defining the body cavity as the trailing region follows along, and wherein the hollow element has a substantially constant diameter therealong upon being everted and fully inflated.

In still another aspect of the present invention there is provided a device incorporating an inflatable hollow element for being positioned along a body cavity of a patient, wherein the hollow element has a leading region and a trailing region and is arranged for being everted progressively along the hollow element from the leading region to thereby be increasingly extended for achieving said positioning in the body cavity as the trailing region of the hollow element follows along, and wherein the hollow element is adapted for limiting swelling during inflation thereof for thereby preferentially causing the trailing region to be drawn along.

Preferably, the device further comprises an enclosure in which the hollow element is located and which is provided with an opening for progressive passage therethrough of the hollow element from the interior of the enclosure, and wherein the interior of the enclosure is sealed from the opening by the hollow element.

The enclosure may comprise an insertion head for being inserted into an entrance of the body cavity and having a through passageway defining the opening. Preferably, the enclosure also comprises a flexible bag sealingly secured to the insertion head.

Preferably, the bag will be adapted for preferentially causing the hollow element to be forced from the enclosure through the opening of the insertion head upon pressure within the enclosure being increased for causing the hollow element to be inflated.

Preferably, the leading region of the hollow element is folded back upon the insertion head such that the hollow element is thereby invaginated.

Preferably, the device will be adapted for enabling coupling of a vibrator thereto for assisting positioning of the hollow element along the body cavity as described above. Preferably, the insertion head will be adapted for being coupled with the vibrator.

The insertion head will also usually be adapted for being inserted directly into the body cavity of the patient with the opening of the through passageway orientated along the body cavity.

Preferably also, the enclosure may incorporate an inlet for entry of a fluid into the enclosure for causing the hollow element to be driven from the enclosure through the opening in the insertion head and thereby everted. The inlet may be defined in the insertion head or the flexible bag comprising the enclosure.

In addition, the device may comprise a pump for pumping the fluid into the enclosure through the inlet. Pressure indicating means may also be provided for indicating pressure exerted on the balloon by the fluid in the interior of the enclosure.

In embodiments not provided with a pump, the enclosure may be at least partially filled with a fluid, and the device further comprise a collar constricting the bag and being slidable along the bag toward the insertion head to force the hollow element to be driven from the opening of the enclosure by the fluid and thereby be progressively everted.

It is not necessary for the hollow element to initially be arranged in an invaginated conformation. Rather, the hollow element may be arranged to assume an invaginated conformation as eversion of the hollow element commences, upon sufficient pressure being applied to the hollow element by the fluid.

The fluid may be any fluid suitable for causing the eversion of the hollow element. Preferably, the fluid will be water or a gas such as air or for instance, nitrogen.

Moreover, one or more indicators for indicating an aspect or aspect of the surrounding environment such as pH, temperature or other physical parameter at a location along the body cavity when the hollow element is positioned therein can be carried on or by the hollow element.

Generally, the hollow element will be an elongate tubular member. The tubular member may be an inflatable balloon, or a tube or stent having a through passageway extending from one end of the tube to an opposite end thereof.

In yet another embodiment, opposed counter rotatable rolls may be used for achieving the eversion and so positioning of the hollow element. As fluid is not used there is also no requirement for an enclosure as described above.

Hence, in another aspect of the present invention there is provided an arrangement incorporating an elongate hollow element for being positioned within a body cavity of a patient, comprising:

a guide head having a through passageway receiving the hollow element; and

at least one pair of opposed counter rotatable rolls for feeding the hollow element through an opening defined by the through passageway of the guide head;

wherein the hollow element has a leading region and a trailing region and is arranged for being everted progressively along the hollow element from the leading region thereof to thereby be increasingly extended from the guide head upon being fed through the opening of the guide head by the counter rotatable rolls.

The counter rotatable rolls will typically be arranged for being separated once the hollow element has been located in position, to allow entry into the interior of the hollow element through an open end thereof.

A hollow element as describe herein will generally have an external diameter of a dimension such that when the hollow element is positioned within the body cavity, the exterior surface of the hollow element presses against the surrounding wall defining the body cavity. Accordingly, embodiments of the invention are particularly suitable for use as a tamponade for applying pressure to sites of bleeding to stem blood flow from the affected site in the management of homeostasis.

The positioning of the hollow element may also be utilised to remove or reduce an intussusception in for instance the gastrointestinal tract of the patient and particularly, in the small bowel.

In addition, the hollow element is particularly suitable for use in angioplasty and in techniques involving positioning or utilisation of stents. In such instances, a device of the invention may in addition to the hollow element comprise delivery means for delivering the hollow element to a location within the body cavity of the patient where the hollow element is then caused to undergo eversion for achieving positioning of the stent within the body cavity. Typically, the delivering means will be a catheter or similar such instrument.

Therefore, in another aspect of the present invention there is provided a method of treating a patient utilising a hollow element for being positioned along a body cavity of a patient and having a leading region and a trailing region, the method comprising:

causing the hollow element to be everted progressively along the hollow element from the leading region thereof such that the hollow element is forced to be increasingly extended and thereby progressively line a surrounding wall defining the body cavity as the trailing region follows along, to thereby effect the treatment.

Preferably, the interior surface of the hollow element will be coated with one or more substances for being delivered to the interior surface of the surrounding wall defining the body cavity with eversion of the hollow element. The substance may, for instance, be an agent for inhibiting blood loss from a bleed site by promoting coagulation, an isotope, or a drug or other therapeutic compound for treating a site or sites along the surrounding wall defining the body cavity.

Accordingly, in a yet further aspect of the invention there is provided a method of applying a substance to a surrounding wall defining a body cavity of a patient utilising a hollow element with an internal surface coated with the substance, wherein the hollow element has a leading region and a trailing region, and the method comprises:

causing the hollow element to be everted progressively along the hollow element from the leading region thereof such that the hollow element is forced to be increasingly extended and the interior surface of the hollow element thereby brought into contact with the surrounding wall of the body cavity as the surrounding wall is progressively lined with the hollow element and as the trailing region follows along;

wherein the hollow element self navigates along within the body cavity following a path determined by the surrounding wall defining the body cavity.

In another aspect of the present invention there is provided a method of locating a stent in position along a body cavity of a patient, wherein the stent has a leading region and a trailing region; and the method comprises;

causing the stent to be everted progressively along the stent from the leading region thereof such that the stent is forced to be increasingly extended and thereby progressively line a surrounding wall defining the body cavity as the trailing region follows along.

In still another aspect of the present invention there is provided a stent for being positioned along a body cavity of a patient, wherein the stent has a leading region and a trailing region and is arranged for being everted progressively therealong from the leading region to thereby be increasingly extended for progressively lining a surrounding wall defining the body cavity as the trailing region follows along.

In yet another aspect of the present invention there is provided a device for use in treating a patient and comprising:

an inflatable hollow element; and

a stent having a leading region and a trailing region and which is arranged for being everted progressively therealong from the leading region to thereby be increasingly extended for progressively lining a surrounding wall defining a body cavity as the trailing region follows along;

wherein the stent overlays the hollow element and the hollow element is arranged in an invaginated conformation for causing the stent to be everted for thereby lining the surrounding wall of the body cavity upon the hollow element being inflated.

In addition, the positioning of a hollow element in accordance with the invention may assist in examination or treatment of the patient by facilitating subsequent insertion of a device such as an endoscope or other instrument into the body cavity upon the hollow element being positioned. Alternatively, the hollow element may be carried on the instrument itself for facilitating insertion and progression of the instrument along the body cavity.

Accordingly, in another aspect of the present invention there is provided a method of examining a patient utilising an elongate hollow element for being positioned along a body cavity of the patient, wherein the hollow element is adapted for facilitating the examination of the patient and has a leading region and a trailing region, and the method comprises:

causing the hollow element to be everted progressively along the hollow element from the leading region thereof such that the hollow element is forced to be increasingly extended and thereby progressively positioned along the body cavity; and

examining the patient.

In yet another aspect of the present invention there is provided a method of locating an instrument along a body cavity of a patient utilising an elongate hollow element, wherein the instrument is attached to the hollow element and the hollow element has a leading region and a trailing region, and the method comprises:

causing the hollow element to be everted progressively along the hollow element from the leading region thereof such that the hollow element is forced to be increasingly extended and the apparatus thereby increasingly drawn along the body cavity as the trailing region follows along, whereby the instrument is located in position within the body cavity.

In a further aspect of the present invention there is provided an apparatus for being inserted into a body cavity of a patient and comprising:

an instrument; and

at least one inflatable hollow element having a leading region and a trailing region, and being secured to the instrument;

wherein the hollow element is arranged for being everted progressively from the leading region thereof while being inflated, to thereby be increasingly extended for progressively lining a surrounding wall defining the body cavity and drawing along the instrument as the trailing region follows along behind.

In another aspect of the present invention there is provided an apparatus for being inserted into a body cavity of a patient and comprising:

an instrument; and

at least one inflatable hollow element secured to the instrument and for being pressed against a surrounding wall defining the body cavity;

wherein the hollow element is arranged for overlying a region of the instrument forward of a location or locations where the hollow element is secured to the instrument, for enabling the hollow element to be progressively everted in a direction along the hollow element upon the hollow element being inflated and the instrument being driven along within the body cavity, for thereby facilitating travel of the instrument along within the body cavity.

The instrument maybe adapted for facilitating collection of a biopsy or a sample from within the body cavity, an instrument for performing a surgical task such as removal of polyps and/or for instance cauterisation of a site as may be required. Typically, the instrument will be an endoscope, colonoscope, fibroscope, gastroscope, laproscope, bronchioscope or other viewing device.

In a particularly preferred device of the invention, an imaging device such as a charged coupled device (CCD) is carried on the hollow element for enabling viewing within the body cavity forward of the hollow element following the positioning of the balloon. In an alternate form, the balloon may carry one or more optic fires for enabling viewing within the body cavity.

Upon deflation of the hollow element, the hollow element can be drawn rearwardly along the body cavity to thereby allow the surrounding wall defining the body cavity to be viewed as the hollow element is withdrawn from the body cavity. This method of examination may also be carried out using apparatus of the invention of the type incorporating a viewing instrument such as an endoscope carrying a balloon for being inflated within the body cavity as described above.

The hollow element may be made of any material deemed suitable for being positioned within the body cavity of the patient and which is capable of being everted in accordance with the invention in order to be positioned therein. Particularly suitable materials include vinyl polymers, latex, polypropylene including ultra high density polypropylene, polyethylene including linear low polyethylene, polyurethane, neoprene and other plastics material having the requisite flexibility.

The device of the invention finds application in both medical and veterinary fields and accordingly, the term “patient” is to be taken to include humans and non-human animals such as those of the ape, equine, bovine and ovine families. Typically, the patient will be a human being.

The body cavity of the patient may be that of the uterus, urethra, ureter, bladder, oesophagus, stomach, a bronchi, a fallopian tube, intestine, colon, or the nasal cavity or gastrointestinal tract of the patient or for instance, a blood vessel, particularly but not exclusively, an artery. Accordingly, while apparatus of the invention is particularly suitable for use in connection with treatment or examination of the gastrointestinal tract and particularly the lower gastrointestinal tract, the invention finds broad application and is not restricted thereto.

The method of the invention enables the hollow element to be inserted relatively quickly and easily, and may assist in reducing patient discomfort, commonly associated with internal treatment or examination of areas such as in the lower gastrointestinal tract. In addition, the hollow element of the device may be inserted without a high degree of training being required and it is not essential when the hollow element is to be used as a tamponade for instance, that the bleed or treatment site be specifically identified and located prior to the positioning of the hollow element. This enables valuable time to be saved in emergencies where it is highly desirable for the well being of the patient for the tamponade to be located in position as quickly as possible. Preferred devices embodied by the present invention are also inexpensive and portable. Advantageously, devices described herein may be made from disposable materials provided for single use such that the device can be simply discarded after use without the need for cleaning and sterilisation.

The invention will now be further described hereinafter with reference to a number of preferred embodiments illustrated in the accompanying drawings.

### BRIEF DESCRIPTION OF THE ACCOMPANYING DRAWINGS

FIG. 1 is a longitudinal cross-sectional view of a rectal inserter;

FIG. 2 is a schematic longitudinal cross-section view of the insertion head of the rectal inserter of FIG. 1 when positioned;

FIG. 3 is a longitudinal partial cross-sectional view of a device embodied by the present invention;

FIG. 4(a) is a longitudinal cross-sectional view illustrating the insertion head of the device shown in FIG. 3 when inserted into the outer holder of the rectal inserter of FIG. 1;

FIG. 4(b) is a side view of another insertion head;

FIGS. 5(a) to 5(d) illustrate the eversion of a balloon of a device embodied by the present invention from an invaginated conformation to an extended conformation.

FIGS. 6(a) to 6(d) illustrates the passage of the outer side of the balloon of the device shown in FIG. 5(a) around a bend in the lower gastrointestinal tract of a patient;

FIGS. 7(a) to 7(d) illustrate the passage of the inner side of the balloon shown in FIG. 6(a) around the same bend;

FIGS. 8 to 12 illustrate further embodiments of the present invention;

FIGS. 13(a) and 13(b) illustrate the eversion of a balloon of another device embodied by the present invention from an invaginated conformation to an extended conformation;

FIGS. 14(a) and 14(b) illustrate the eversion of a balloon of a yet further device embodied by the present invention from an invaginated confirmation to an extended conformation;

FIGS. 15(a) to 15(c) illustrate the positioning of a yet further device embodied by the present invention involving eversion of a balloon of the device from an invaginated conformation to an extended conformation to enable further progression of the device along a body cavity of a patient;

FIGS. 16 and 17 illustrate hollow elements having invaginated conformations and which are useful for facilitating angioplasty and positioning of stents along the body cavity of a patient;

FIG. 18 illustrates the hollow element of FIG. 11 or 12 when in an extended conformation;

FIG. 19 illustrates the hollow element of FIG. 18 in position along a body cavity of a patient;

FIGS. 20(a) and 20(b) illustrate the use of a plunger to cause a hollow element of a device embodied by the invention to undergo eversion;

FIG. 21 illustrates a yet further device embodied by the invention in which a stent is carried on a hollow element of the device;

FIGS. 22(a) and 22(b) illustrate yet another device embodied by the present invention; and

FIG. 23 is a diagrammatic front view of the balloon arrangement illustrated in FIG. 22.

### DETAILED DESCRIPTION OF PREFERRED EMBODIMENTS OF THE INVENTION

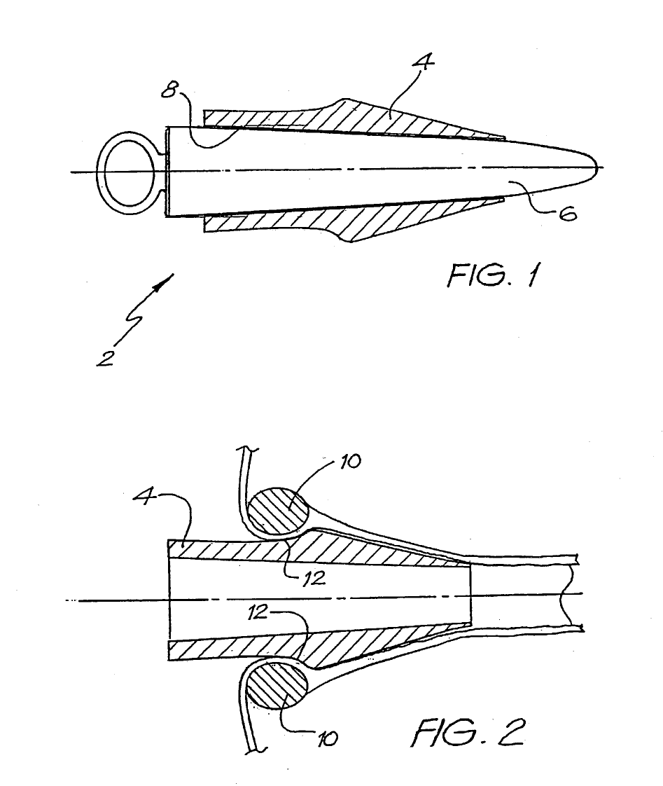

The rectal inserter 2 shown in FIG. 1 comprises an outer holder 4 and a core insert 6 received in a through passageway 8 extending from a forward end of the outer holder to an opposite end thereof. The outer holder is tapered to facilitate insertion into the rectal opening of a patient. The core insert 6 is used to press the outer holder 4 into the rectum prior to being subsequently withdrawn once the outer holder is held in position by grip exerted by the anal sphincters 10 around the recessed area 12 of the outer holder. As can be seen, the outer holder 4 of the rectal inserter acts to allow access to the lower gastrointestinal tract when in position.

The device 14 shown in FIG. 3 comprises a hollow tapered insertion head 16 formed from stainless steel, and a flexible plastic bag 18 indicated in phantom outline. The open end of the bag 18 is sealed around the rear end 20 of the insertion head and is securely fixed thereto such that the insertion head 16 and bag 18 together comprise an enclosure in which is received a hollow element in the form of a deflated balloon 22. The bag 18 is shown substantially truncated for explanatory purposes. The bag, however, is sufficiently long to allow the balloon to extend out to its full length.

A leading region 24 of a hollow element in the form of balloon 22 extends from opening 26 of the insertion head 16 and is folded back around the insertion head such that the balloon 22 is arranged in an invaginated conformation and the interior 28 of the balloon opens to the exterior of the inserter head through the opening. The leading region of the balloon is fixedly secured around the insertion head 16 by a ring clamp (not shown) such that the interior of the insertion head and the enclosure as a whole is sealed from the interior of the balloon. In alternative embodiments, a circumferential groove may be defined in the outer face of the insertion head and the balloon secured in position by an O-ring or other suitable means received in the groove to thereby fixedly clamp the balloon to the insertion head.

A squeezable hand pump 30 is connected to inlet 32 of the insertion head 16 through flexible tubing 34. A pressure gauge 36 is arranged in-line in the flexible tubing for indicating the pressure of air within the interior of the insertion head upon being pumped therein with operation of the hand pump 30 in use. The hand pump 30 is provided with a pressure release valve 38 to release air and thereby lower and have regulate pressure within the insertion head 16 as required. Rather than a hand pump, any suitable mechanical or electrical pump may of course be employed.

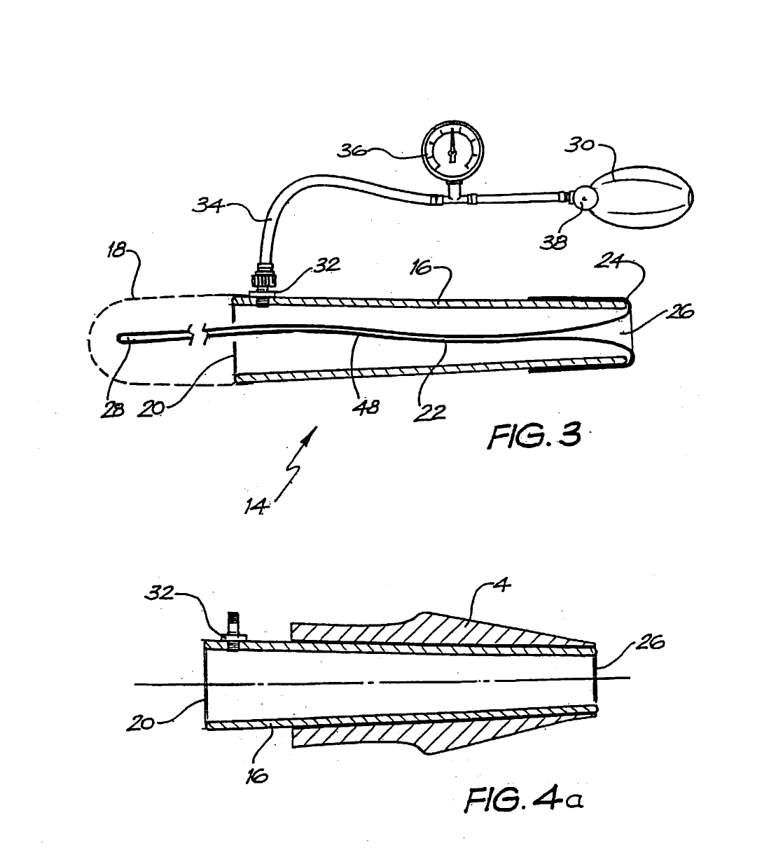

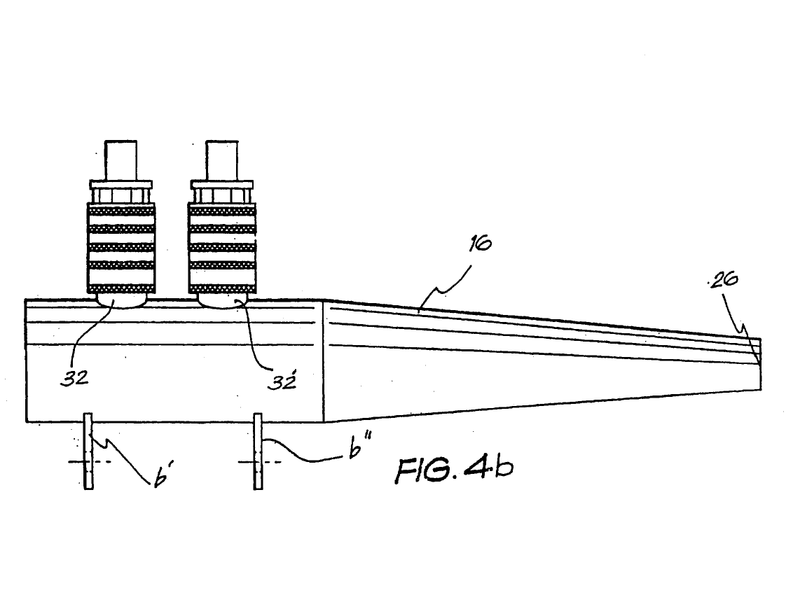

In use, the insertion head 16 is located in the outer holder 4 of the rectal inserter 2 when positioned in the rectum of a patient, as generally indicated in FIG. 4(a) (the balloon 22 and bag 18 are not shown). Alternatively, an insertion head adapted for being inserted directly into the rectum may be utilised in the absence of an outer holder 4. A further insertion head is illustrated in FIG. 4(b). As can been seen, the insertion head is provided with nipples 32 and 32′ comprising an inlet and outlet respectively, for the passage of fluid into and from the enclosure head. Adaptors are fitted to the nipples for the reception of hoses for the supply and return of fluid to and from the interior of the insertion head. As will be understood, a pressure release valve or pressure regulator may be arranged in or in connection with the return hose for regulating pressure within the insertion head and specifically, for maintaining the pressure at or below a predetermined upper limit.

The insertion head shown in FIG. 4(b) is also provided with brackets b′ and b″ for facilitating coupling of a vibrator (not shown) to the insertion head for facilitating travel of the balloon along within the lower gastrointestinal tract as will be further described below. The vibrator may be any conventional device and may be fixed to the brackets via a suitable mounting holding the vibrator.

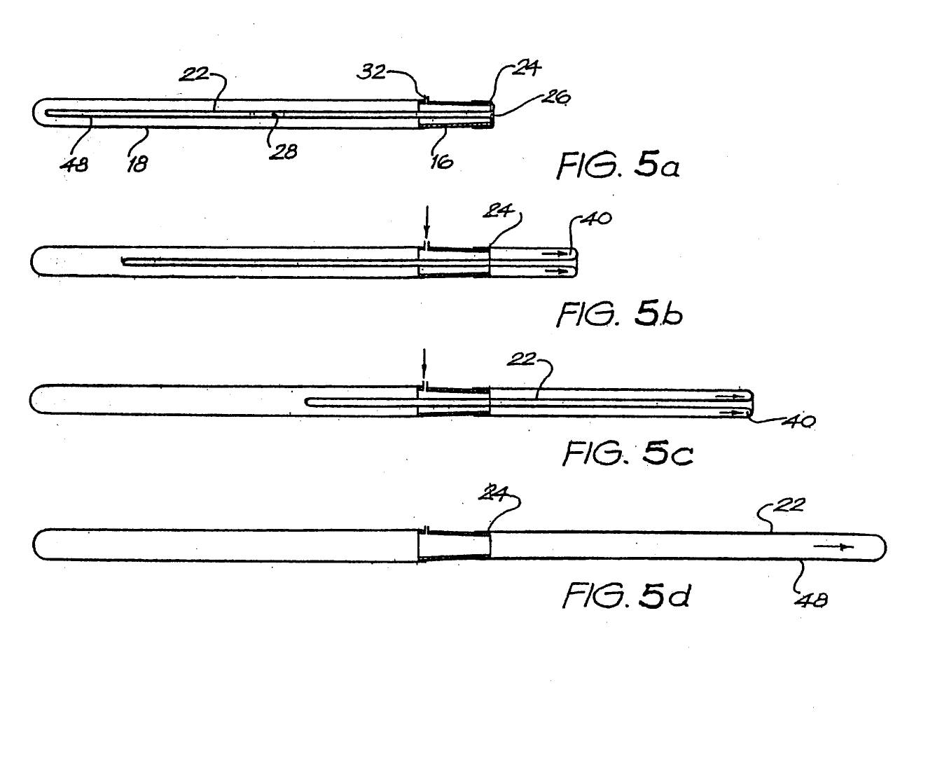

The operation of the device to cause the balloon to be extended from the opening 26 of the insertion head 16 will now be described with reference to FIGS. 5(a) to 5(d).

Upon entry of air into the insertion head 16 through the inlet 32 with operation of the hand pump 30, pressure is applied to the annular base of the invagination in the balloon indicated by the numeral 40. With further operation of the hand pump 30, the applied pressure increases until sufficiently high such that the balloon is forced through the opening 26 of the insertion head 16. As the leading region 24 of the balloon 22 is secured to the insertion head 16, the balloon is caused to the everted progressively from the leading region of the balloon with passage of the balloon through the opening 26 as indicated in FIGS. 5(b) and 5(c). With continued entry of air into the interior of the insertion head, the balloon 22 is caused to be fully extended as indicated in FIG. 5(d). In this way, the balloon may be progressively inserted into the lower gastrointestinal tract of the patient so as to lie therealong. The balloon is not restricted to being inserted into a relatively linear region of the lower gastrointestinal tract and is capable of passage around bends having significant curvature. As can be seen in FIG. 5d, the balloon has a substantially constant diameter thereablong when in the fully extended and inflated condition.

As will be understood, the bag 18 is formed from a material that is significantly less stretchable compared to that from which the balloon 22 is made, thereby facilitating the forcing of the balloon through the insertion head. Desirably, stretching of the bag will be minimal upon the bag being inflated for causing extension of the balloon from the insertion head.

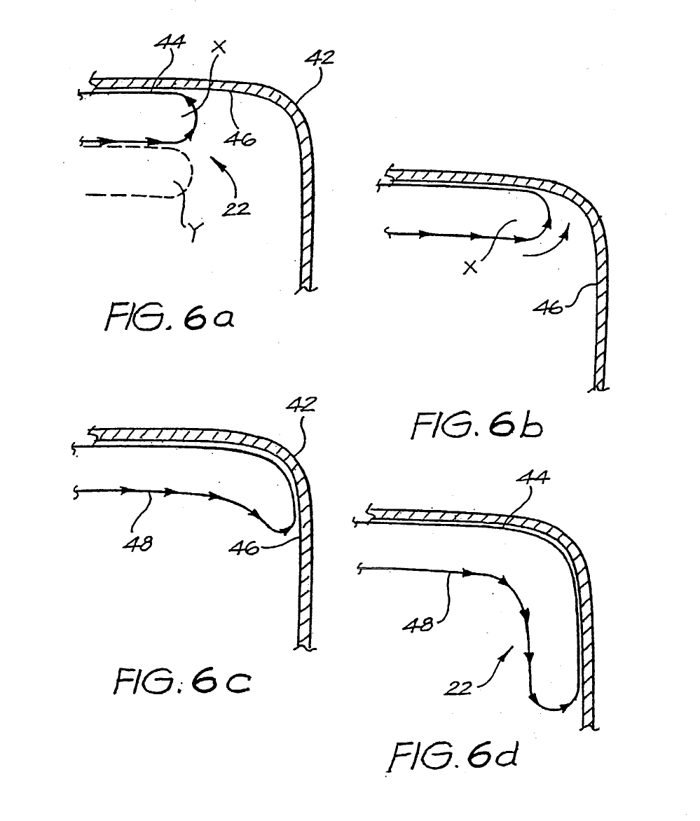

Travel of the balloon around a bend 42 in the colon is illustrated sequentially in FIGS. 6(a) to 6(d). For explanatory purposes, only the progression of the outer side X of the balloon is shown. The opposite inner side Y of the balloon is indicated in FIG. 6(a) in phantom outline. As can be seen, the surface 44 of the balloon in contact with the interior surface 46 of the colonic wall remains stationary as the trailing region of the balloon 48 is drawn along and directed into contact with the colonic wall as air enters the balloon with operation of the hand pump. Accordingly, the balloon effectively tracks along the colon as the balloon undergoes eversion. The direction of movement of the trailing region of the balloon is indicated by the arrows.

Upon reaching bend 42 in the colon, the outer side X of the balloon is directed therearound by the curvature in the colonic wall.

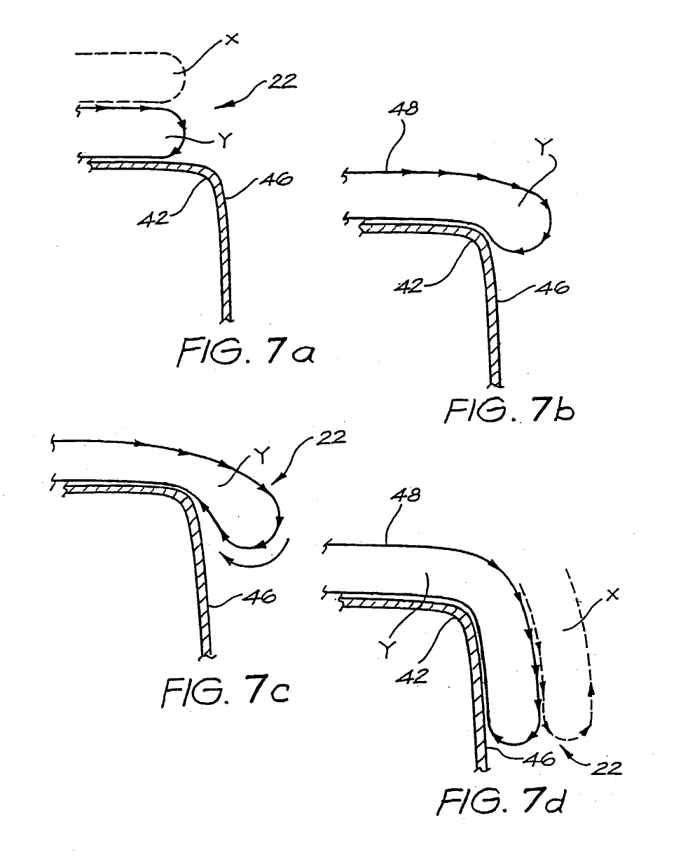

Travel of the opposite inner side Y of the balloon around the bend 42 of the colon is illustrated in FIGS. 7(a) to 7(d). In this instance, the inner side of the balloon leaves the colonic wall for a distance until the balloon is around the bend at which time it is pressed back into contact with the colon by the opposite outer side of the balloon as indicated in FIG. 7(d).

The movement of the balloon around the bend is facilitated by bulging of the unfolding balloon as a result of the balloons elasticity which assists in redirecting the balloon, together with the continuous feed of the trailing region of the balloon into the open interior region of the colon. The bulging described above may be facilitated by using a balloon having an outer diameter that is larger than the interior diameter of the colon. Desirably, the material from which the balloon is formed while being resilient should not be excessively stretchable such that upon negotiating the bend, bulging of the unfolding balloon front and swelling of the balloon in general is restricted resulting in the trailing region of the balloon being preferentially drawn along.

Generally, the balloon will be inflated to a pressure of between about 40 mmHg for causing the progression of the balloon along a relatively straight path. The pressure may be increased to about 80 mmHg or more for facilitating passage of the balloon around a bend. The pressure to which the balloon is inflated will of course depend on the particular application in which the apparatus is being utilised and the material from which the balloon is made, and can be readily determined in accordance with routine trial and experimentation. Desirably, pressure in the balloon will be maintained at or below a determined maximum pressure for the particular application and apparatus utilised.

As discussed above, a vibrator may be coupled to the insertion head. When operated, vibrations from the vibrator may be transmitted along the balloon to the unfolding balloon front and assist progression of the balloon and particularly, around bends encountered.

It is not necessary that the interior of the colon be clear or otherwise free of obstructions in order for the balloon to be able to progress along within the colon. More particularly, when the balloon contacts an obstacle such as a stool, the balloon is caused to be lifted from the interior surface of the colonic wall and track over the obstacle by the pressure of the air within the bag resulting from continued operation of the pump. Advantageously, the obstacle is not pushed along by the balloon to any significant degree and is passed over by the balloon substantially without being displaced. As the eversion of the balloon causes opposite sides of the colonic wall to be parted as the balloon approaches, the balloon is also able to travel along substantially without longitudinal displacement or stretching of the colonic wall itself to any significant degree.

Generally, the balloon is capable of passing through regions of the colon or other section of the lower gastrointestinal tract the internal diameter of which is restricted by greater than 50% compared to adjacent regions by a stenosis and more preferably, by about 75% or more. Indeed, the balloon may be capable of passing through a number of stenosies that restrict the inner diameter of the colon to varying degrees.

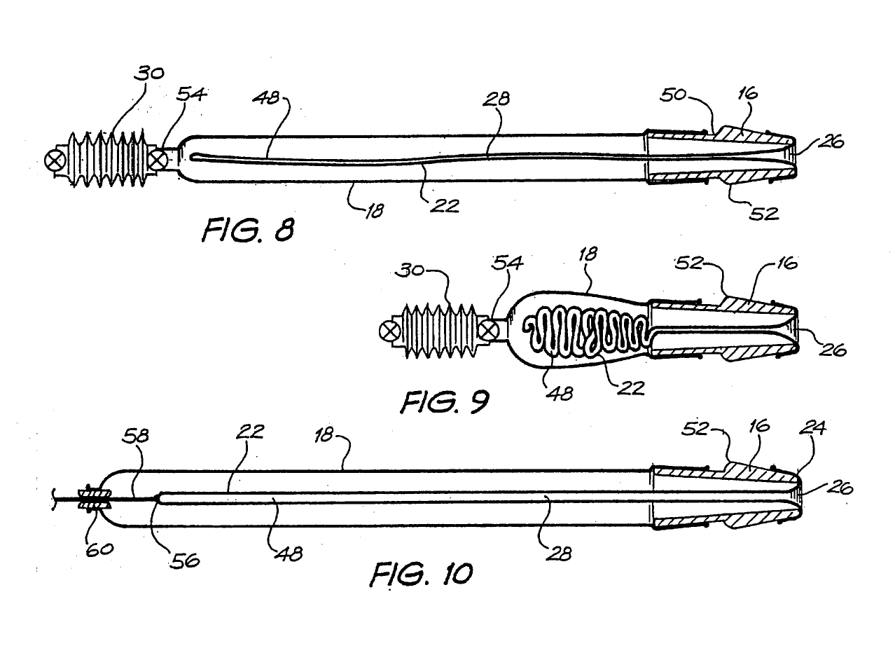

A further embodiment of the present invention is illustrated in FIG. 8. This device differs in that the insertion head 16 is adapted for being inserted directly into the rectal opening of the patient without the need for use a rectal inserter 2. More particularly, the insertion head is tapered in a direction from the front of the insertion head to the rear thereof, and is recessed 50 behind raised annular rim 52 providing a section for being gripped under the action of the anal sphincters to inhibit withdrawal of the insertion head 16 in the same manner as the rectal inserter 2 shown in FIG. 2. The hand pump 30 is also arranged to pump air directly into bag 18 through inlet 54 rather than though the insertion head.

In the device shown in FIG. 9, the balloon 22 is in a concertinaed conformation enabling the length of the bag 18 to be significantly reduced. This device functions on the same principle as that shown in FIG. 8 in that the balloon is progressively forced through the opening 26 of the insertion head 16 upon air pumped into the bag 18 by the hand pump 30 reaching sufficient pressure, and is thereby caused to undergo eversion as the balloon is increasingly extended. With inflation, the balloon assumes a substantially straight form. Upon subsequent deflation, the balloon resumes its concertinaed conformation assisting in withdrawal of the balloon from the patient.

The balloon can be deflated by operation of the pressure release valve of the pump and be withdrawn from the patient by being pulled rearwardly by the medical attendant or physician following the deflation of the balloon.

Withdrawal of a balloon may also be assisted by the provision of one or more cords or the such like attached to the exterior of the end 56 of the balloon as shown in FIG. 10. In this embodiment, a single cord 58 is shown secured to the balloon 22 and which passes from bag 18 through a seal 60 for enabling the cord to be gripped. Any suitable seal arrangement conventionally known in the art may be utilised. The seal may for instance, comprise a resilient plug that sealingly grips the cord as the cord passes therethrough. The plug may be integrally formed with the bag or be heat welded or otherwise securely fixed to the bag. Upon inflation of the balloon, the string is drawn through the seal 60 as the balloon is forced from opening 26 of the insertion head 16. The seal 60, while allowing movement of the string therethrough acts to inhibit loss of air from the bag 18 and thereby maintain air pressure as the balloon is being inflated, and when it is fully inflated.

To withdraw the balloon from the patient, the balloon can either be allowed to entirely deflate prior to the cord 58 being pulled while the insertion head 16 is also held to prevent it being tugged from the rectum under the pulling action or alternatively, pressure within the enclosure formed by the bag 18 and insertion head 16 can be reduced gradually as the cord 58 is pulled such that the extended balloon 22 is progressively pealed from the colonic wall. In this way, the balloon is removed by a process that is substantially the reverse of that by which the balloon was located in position, and significantly reduces friction exerted on the interior surface of the colonic wall and the potential risk of abrasion during the removal of the balloon. This later method of withdrawal is particularly preferred when the balloon has been inserted a significant distance into the patient, or extends around a bend or bends upon being positioned within the patient.

If desired, electrically or mechanically operated pumps may be used to inflate the balloon rather than a hand pump 30. Other means for causing the balloon to be positioned in the body cavity may also be used. For instance, in the embodiment shown in FIG. 11, the device is provided with a slidable collar 62 receiving the bag 18 of the device. The internal diameter 64 of the collar is dimentioned to tightly squeeze the bag 18 to inhibit leakage of sterile water with which the forward region 66 of the bag is filled to the rear region 68 of the bag, as the collar is slid along the bag toward the insertion head 16 while the rear region 68 of the bag is held. As will be appreciated, the travel of the collar along the bag 18 causes the water to exert pressure on the balloon 22 and force the balloon from the insertion head 16 in use. Deflation of the balloon can be achieved simply by withdrawing the collar along the bag 18.

In another embodiment, balloon 22 may be received within a second balloon both of which are initially arranged housed within the enclosure defined by the insertion head 16 and the bag 18 in an invaginated conformation, the second balloon being substantially more elastic than balloon 22. Upon the pressure within the bag 18 being increased, the second balloon is caused to be progressively everted from within the enclosure through the insertion head therealong and extended by balloon 22. Once the balloons have been extended, the second balloon is selectively inflated to apply pressure to the surrounding wall defining the body cavity, preferably while the balloon 22 is retained in an inflated condition. Moreover, the second balloon may be coated with a drug, isotope or other substance for application to the surrounding wall. As will be understood, the mouth of the second balloon is secured around the insertion head in the same manner as balloon 22, but is able to be inflated independently of balloon 22 as indicated above. This may be achieved by arranging the mouth of the second balloon over an outlet defined in the insertion head through which a fluid, preferably air or nitrogen for instance, may be pumped into the second balloon from pump 30 through a dedicated passageway defined in insertion head that terminates in the outlet, and which is sealed from the interior of the enclosure and communicates with the pump via a separate hose leading from the insertion head, the entry of air into the second balloon being controlled by a valve arranged in that hose or otherwise controlling flow through that hose.

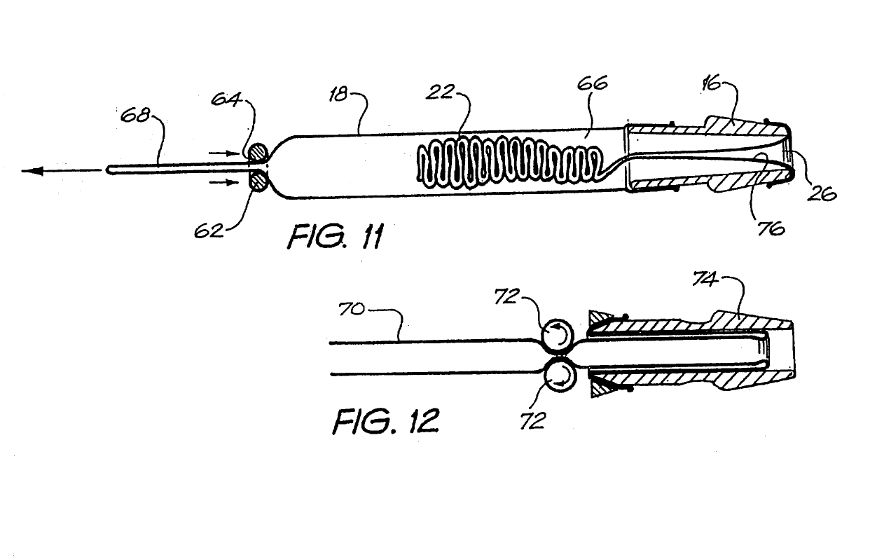

In FIG. 12, there is shown an arrangement for achieving insertion and thereby the positioning of a hollow tube 70 open at each end. This device is provided with a pair of opposed, counter-rotatable rolls 72 which grip the tube as the tube is fed through the nip of the rolls causing the tube to be driven along through the head 74 and so be everted in the same manner as other embodiments described above. The insertion head 74 is indirectly coupled to the rolls and may be adapted for being inserted directly into the patient or alternatively, for being received in a separate dedicated holder or inserter. The embodiment shown in FIG. 12 also differs in that no fluid is used to cause eversion or inflation of the tube. In this instance, the tube 70 has an inherent inflated conformation and is formed from a material having sufficient resilience to return to that conformation upon passing through the nip of the rolls 72 such that the tube is thereby able to act in the same manner as the balloon 22 of the above described devices.

Withdrawal of the tube from the patient can be achieved by reversing the rotation of the rolls such that the tube is removed in a substantially reverse process to that by which it was inserted into the patient. The tube may also be inserted to simply provide an open passageway along the body cavity in which it is inserted for subsequent passage of an endoscope, colonoscope, fibroscope or other instrument through the tube. In this instance, the rolls may be separated to allow entry of the viewing instrument into the interior of the tube once the tube has been positioned.

The hollow element of the above described devices applies pressure to the interior surface of the surrounding wall defining the body cavity in which it is inserted. Accordingly, the device is particularly suitable for use as a tamponade such as in the management of colorectal bleeding and bleeding at other sites within the lower gastrointestinal tract through the application of pressure to the bleed site by the hollow element. As the balloon is capable of extending along the lower gastrointestinal tract some distance it is not necessary that the site of the bleed be identified prior to insertion of the hollow element.

If desired, the interior surface 76 of the hollow element may be coated with an effective amount of one or more suitable substances for inhibiting or preventing further bleeding from the affected site upon being brought into contact with the site as a result of the eversion of the balloon while being positioned. Such substances include coagulants such as thrombin or thrombogenic substances. If appropriate, at least limited regions of the interior surface of the hollow element may be coated with a sclerosant or thrombosant agent for causing sclerosis or scarring to inhibit recurrent bleeding.

Similarly, the device may be used as a means of delivering therapeutic or other substances in general, including radioisotopes, steroids and drugs to specific sites of the surrounding wall defining the body cavity. Such sites include sites of disease such as cancer within the lower gastrointestinal tract or elsewhere. Examples of drugs include those conventionally used in the treatment of colorectal and other cancers.

The device of the invention may also be used to clear or reduce an intussusception by exerting contact pressure on the intussusception as the hollow element undergoes eversion during the insertion thereof. Such use may significantly reduce discomfort to the patient compared to that arising from the use of conventional pneumatic intussusception reduction techniques.

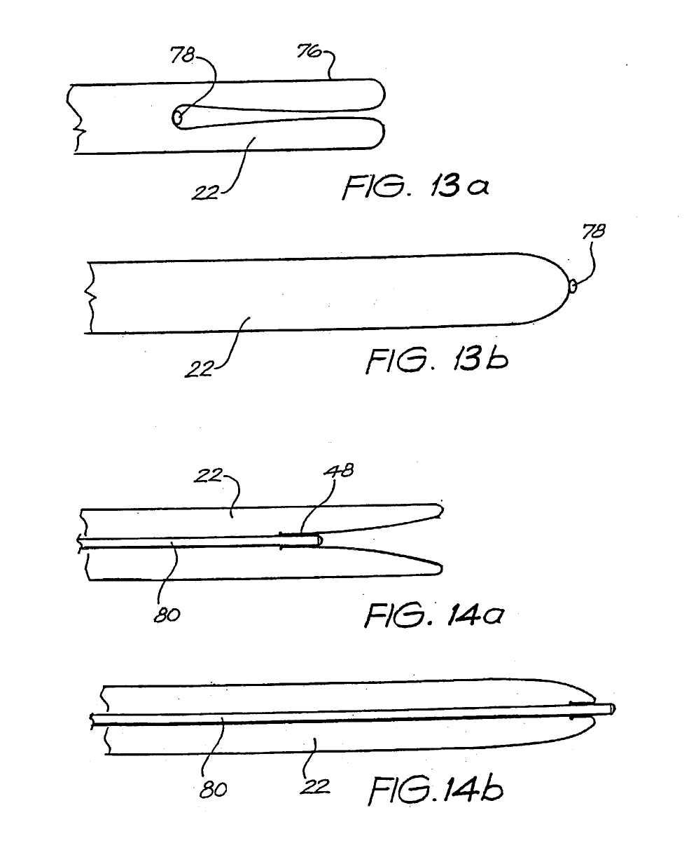

Turning now to FIG. 13(a), there is shown a balloon 22 carrying an imaging means 78 in the form of a charged coupled device (CCD), the CCD being positioned such that when the balloon is fully inflated as shown in FIG. 13(b), the CCD 78 is located for enabling imaging of forward regions of the body cavity in which the balloon is located in use. The CCD 78 is arranged in a casing with a lens (not illustrated) for focusing incident light onto the CCD for generation of a signal which is communicated to external display means, for example a monitor, television or the like. The signal provided by the CCD 78 is preferably digital, although other formats such as analogue signals may be used. In one embodiment the signal is communicated to the display means by conductors such as electrical wires or an optical fibre carrying a modulated signal.

The conductors are arranged to be drawn along as the balloon is everted during the insertion of the balloon, and pass from the enclosure defined by the insertion head 16 and flexible bag 18 as described above, through a dedicated seal as with the cord 58 of the embodiment shown in FIG. 10. However, it is not necessary that the conductors be arranged in this way and can be affixed to surface 44 of the balloon so as to extend along the outside of the balloon when the balloon is in an extended conformation upon being everted.

Other embodiments include wireless transmission means, for example a miniature radio transmitter, which is adapted to receive a signal from the CCD and transmit said signal through the body of the patient to a receiver located externally of the patient. This embodiment advantageously dispenses with the requirement for electrical conductors to connect the CCD to the display means.

A light source (not illustrated) is preferably disposed proximate the CCD, or is integral therewith, so as to illuminate the region of the body cavity to be imaged. In exemplary embodiments the light source takes the form of a miniature globe, a diode, or for instance an optical fibre positioned so as to communicate light from an external light source.

Rather than utilising a CCD, visualisaton within the body cavity may be achieved utilising one or more optic fibres arranged in the same manner as the electrical conductors associated with the CCD. Preferably, a bundle of optic fibres will be used. Generally, the optic fibres will terminate within the balloon when in the extended conformation in which case the balloon, or at least a window at the tip of the balloon, will be transparent to allow viewing into the body cavity through the balloon. However, the optic fibres may sealingly pass through the balloon prior to terminating in a sealed casing sealingly affixed to the exterior of the balloon.

Again, the distal ends of the optical fibres are arranged with one or more lenses held in the casing, for feeding light incident upon the lens into the fibres. The light is transmitted by means of internal reflection along the length of the optical fibre to proximal ends. Light processing means disposed at the proximal ends of the optical fibres receive the light and derive therefrom an image for display upon the display means in a manner known to those skilled in the art. In embodiments as described, a transparent protective shield may overlay the casing in which the or each lens and the CCD or optic fibres are housed for thereby minimising contamination. The shield may comprise for instance, a film of suitable plastics material welded or otherwise affixed to the balloon.

In a similar manner to that described above, a light source is preferably disposed proximate the distal ends of the optical fibres. Various light sources known to those skilled in the art may fulfil this function, including those mentioned above. In one embodiment, a single bundle of optical fibres serves as both the imaging means and the light source. More particularly, a first sub-set of optical fibres are used to communicate light from an external light source which is then used to illuminate the region of the patient to be imaged. This light then reflects from the region and is collected by the distal ends of a second sub-set of optical fibres which communicate the light to the light processing means.

Moreover, one or more indicators for indicating an aspect or aspects of the surrounding environment such as pH or temperature at a location along the body cavity can be carried by the balloon.

An indicator for providing information about pH for instance, may simply be any suitable conventionally known substance or material that undergoes a colour change upon contact with fluid within the body cavity of the patient that can be visually detected upon the removal of the balloon and compared to reference colours to allow an evaluation of the pH to be made.

Alternatively, an indicator may comprise a probe arranged for being exposed to the environment of the body cavity at the location of interest and to send signals to suitably calibrated exterior apparatus for displaying or otherwise indicating information about a given parameter, and with which the probe is in communication through electrical conductors as in the embodiment shown in FIGS. 13(a) and 13(b).

In another embodiment, the trailing region of a balloon may be sealed around an endoscope or other viewing instrument for examining the patient. The insertion of such a device is shown sequentially in FIGS. 14(a) and 14(b). In this instance, the leading region of the balloon is again folded back over an insertion head 16 as generally indicated in FIG. 3. As with that embodiment, a flexible bag is sealingly secured around the rear end of the insertion head. In contrast however, the rear of the bag is sealingly clamped around a region of the endoscope distal from the leading end 82 of the endoscope, such that inflation of the bag causes the balloon to be extended from the insertion head in the manner described above, but in this case, drawing the endoscope along behind. That is, the rear of the endoscope remains external of the bag for allowing operation of the endoscope when the balloon has been located in position within the body cavity. The use of the balloon 22 assists not only in the insertion of the endoscope 80 but also assists in protecting the endoscope from contamination. In addition, the balloon may be readily removed from the endoscope following withdrawal from the patient and so also minimises the cleaning necessary for enabling subsequent use of the endoscope.

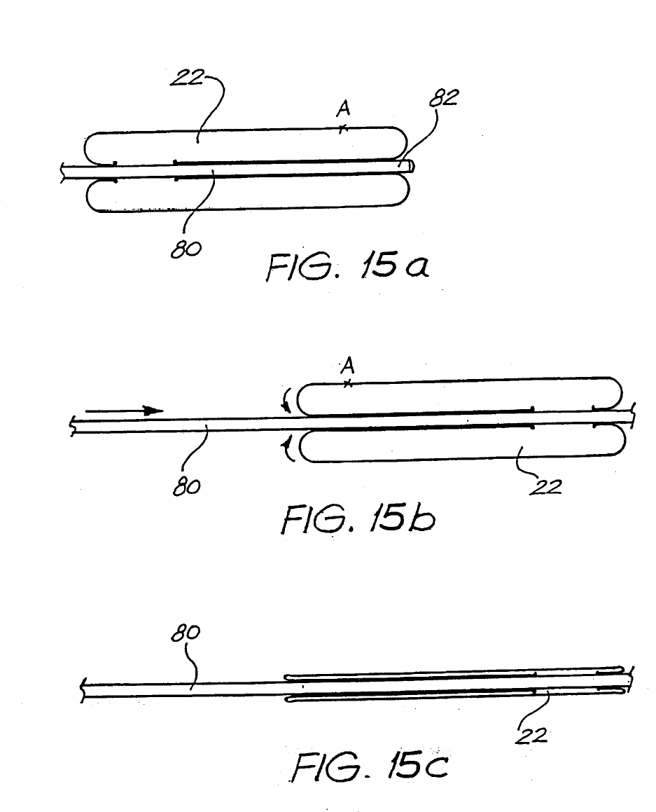

A different form of this type of device is shown in FIGS. 15(a) to 15(c). In this embodiment the balloon 22 is carried entirely on the forward end region of the endoscope 80. Prior to inflation, the balloon is arranged in an invaginated conformation and held tightly against the exterior surface of the endoscope. This is achieved by creating a vacuum within the balloon by withdrawing air therefrom through an aperture defined in the exterior of the endoscope in communication with a channel extending along within the endoscope to external tubing leading to a vacuum pump. Inflation of the balloon as indicated in FIG. 15(a) can be achieved by pumping air into the balloon through the same channel in the endoscope or by a separate such channel that opens into the interior of the balloon through the same aperture as that from which air is withdrawn from the balloon, or a separate aperture.

When the balloon 22 is in the inflated extended conformation, the body cavity within which the device is located is also opened thereby facilitating visualisation forward of the endoscope. Moreover, upon the endoscope 80 being pressed into the body cavity the balloon is caused to track along the endoscope rearwardly from tip 82 as indicated in FIG. 15(b), enabling the endoscope to progress a distance along the body cavity to facilitate the examination as required. The balloon may also be used to negotiate passage of the endoscope around a difficult bend that might not otherwise have been possible in the absence of the balloon or may have caused excessive discomfort to the patient in the absence of the use of the balloon.

The endoscope may carry one or more implements for collecting a biopsy, removing polyps and/or for instance, cauterising a site at a location along the body cavity as is known in the art.

In the above embodiment, rather than a vibrator being secured to the insertion head of apparatus such as indicated above in relation in FIG. 4b, a vibrator device may be arranged within the forward region of the endoscope for vibrating the balloon and endoscope to assist progression of the endoscope along within the body cavity.

The resilience of the material from which the balloon of this embodiment is made may be such that, or the balloon is otherwise inherently adapted such that, the balloon is caused to be drawn at least partly back to its initial position relative to the endoscope as shown in FIG. 15(a) from that shown in FIG. 15(b) as the balloon is deflated, allowing for the possibility of the balloon to be reinflated and the endoscope to be progressed some distance further along the body cavity upon repeating the above steps sequentially one or more times.

When the endoscope is to be withdrawn, the balloon is simply deflated and drawn tightly against the exterior wall of the endoscope upon withdrawal of air from the balloon under vacuum. Examination may be continued as the device of any one of FIGS. 13 to 15 is withdrawn from the body cavity of the patient following deflation of the balloon allowing a reverse endoscopy or examination to be achieved.

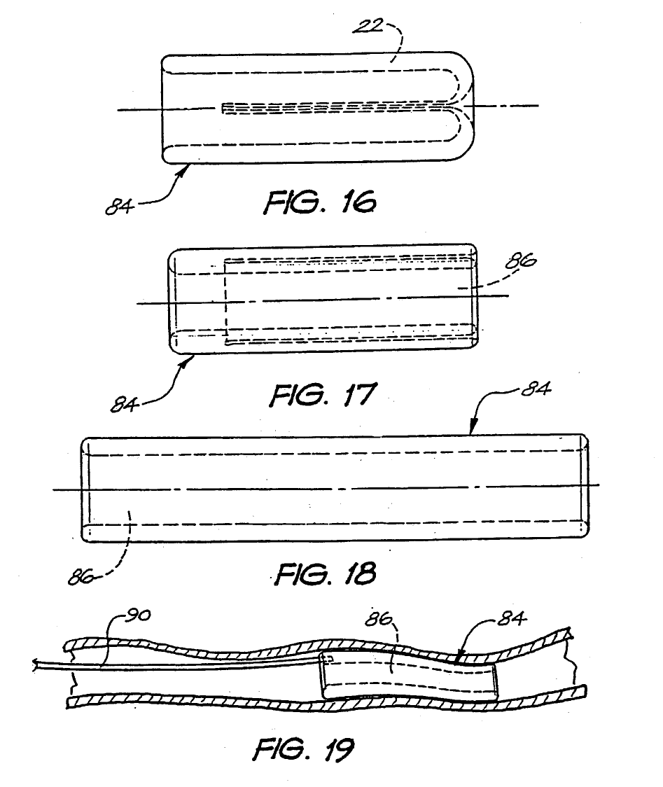

In FIG. 16, there is shown an invaginated hollow element 84 for being located in position along a blood vessel of a patient in an angioplasty technique. A further invaginated such tube is shown in FIG. 17. Each tube consists of a hollow balloon having a through passageway 86 extending from one end of the balloon to an opposite end thereof. Location of the tube within a blood vessel and its inflation to thereby cause the tube to undergo eversion to an extended conformation and be pressed against the surrounding blood vessel wall as shown in FIG. 18 to effect angioplasty, is achieved with the use of a catheter 90 that sealing enters the tube as indicated in FIG. 19.

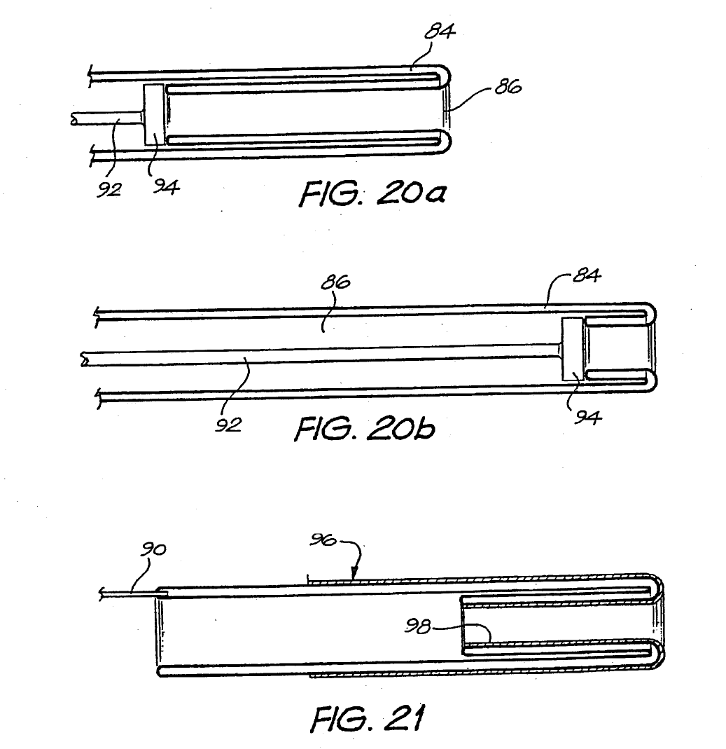

To assist eversion of a tube of the type shown in FIG. 16, a plunger 92 may be used to push the trailing region of the tube along as shown in FIGS. 20(A) and (b). Typically, the plunger head 94 will be adapted for avoiding blockage of the through passageway of the tube and so will usually be provided with at least one passageway extending through the head for the passage of fluid past the head.

However, it is not necessary that an inflatable type tube be used for angioplasty. More particularly, a plunger 92 may be utilised to cause an invaginated tube to be everted without inflation of the tube by air or other fluid. This type of tube will generally have an outer diameter dimensioned such that the tube bears against the blood vessel wall as the tube is everted by the progression of the plunger along the through passageway of the tube. Usually, the tube will be restrained against being driven along the blood vessel while undergoing eversion, by a catheter gripping the tube.

In this embodiment, the tube may be a stent for being permanently left in position within the blood vessel upon being released by the catheter. The stent can be formed from a suitable metallic mesh of the type used in expandable type stents known in the art.

Alternatively, the stent can be deployed within the blood vessel using an inflatable tube as illustrated in FIG. 21. As can be seen, the stent 96 overlays and thereby receives the tube and both the stent and the tube are invaginated, with the trailing region 98 of the stent being folded into the corresponding region of the tube. Accordingly, upon being inflated, the outer diameter of the stent expands until the stent is pressed against the surrounding blood vessel wall, and both the tube and the stent are simultaneously caused to be increasingly extended along the blood vessel as the trailing region of each is drawn along while being progressively everted.

Once the stent is fully extended, the tube can be deflated and withdrawn from within the stent by the catheter used to inflate the tube and initially locate the stent within the blood vessel, leaving the stent behind in the desired position in a fixed expanded conformation.

Yet a further embodiment of the present invention is illustrated in FIG. 22. In this embodiment a miniscope 100 is carried by the balloon 22 for enabling examination upon being positioned within a body cavity of the patient such as the lower gastrointestinal tract following inflation and concomitant eversion of the balloon in the manner above described. The miniscope 100 in turn carries a positioning balloon 102 on a rear end region 104 thereof, and a balloon arrangement 106 on a leading end region 108 of the miniscope for selectively orientating the leading end region in a desired direction as will be described further below.

The miniscope 100 itself is telescopic and is extendable from a retracted position to an extended position as indicted in phantom outline in FIG. 22(b). More specifically, the miniscope incorporates a hydraulic piston system operable to cause the extension and retraction of the miniscope between the extended and retracted positions. The miniscope also incorporates at least one servomotor and associated control system for controlling bending of the leading end region 108 as desired to selectively orientate the viewing tip 110 within the body cavity by the operator. Typically, the miniscope will incorporate at least two such servomotor control systems for facilitating movement of the viewing tip in sideways and directions perpendicular thereto, respectively. Both the hydraulic and servomotor control systems are known in the art in for instance conventional colonoscopes. The required hydraulic lines and electrical conductors serving the servomotor and hydraulic systems are arranged within the balloon 22 and drawn behind the miniscope during the eversion of the balloon as is an optic fibre bundle for enabling viewing through the viewing tip 110.

The positioning balloon 102 is annular in form and encircles the rear end region of the miniscope 100. Accordingly, upon being inflated, the positioning balloon acts to open the body cavity by pressing against the surrounding wall defining the body cavity, and centre the miniscope within the body cavity.

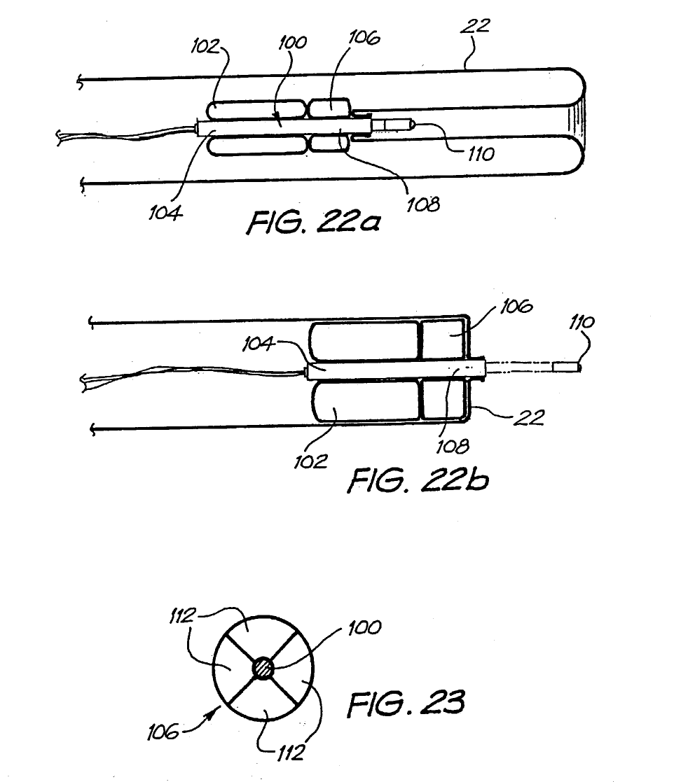

The balloon arrangement 106 comprises a plurality of balloon segments 112 located around the miniscope as is generally indicated in FIG. 23 for being selectively inflated and deflated, respectively. By inflating one or more of the balloon segments and leaving others deflated, the leading end region of the miniscope is forced in a direction toward the deflated segments. With inflation of the previously deflated balloon segments and deflation of the previously inflated ones, the leading end region of the miniscope can be moved in the opposite direction. Fine control is achieved by only partially inflating or deflating ones of the segments. Generally, at least four such balloon segments will be located equidistant around the miniscope as shown although more may be provided if desired. Besides acting to orientate the miniscope 100, the balloon arrangement 106 also assists in opening the balloon cavity forward of the miniscope upon different ones of the segments being inflated and so thereby further assist in the internal examination using the miniscope.

As will be appreciated, at least the leading end region 108 of the miniscope is relatively flexible and the miniscope itself incorporates passageways as required for enabling selective inflation and deflation of not only the positioning balloon 102 but also the balloon arrangement 106. The vacuum and air supply lines also trail along within the balloon for convenience. As with the endoscope embodiment shown in FIG. 14(A), the balloon may for instance be sealingly clamped to the instrument by ring clamps or other suitable clamping devices and/or otherwise by welded or fixed to the instrument by a suitable adhesive.

Accordingly, although the present invention has been described hereinbefore with reference to a number of preferred embodiments, the skilled addressee will understand that numerous variations and modifications are possible without departing from the scope of the invention.

The claims defining the invention are as follows:

1. A method of positioning an elongate hollow element along a body cavity in a patient, wherein the hollow element has a leading region and a trailing region, and the method comprises:
causing the hollow element to be everted progressively along the hollow element from the leading region thereof such that the hollow element is forced to be increasingly extended and thereby progressively line a surrounding wall defining the body cavity as the trailing region follows along; and
wherein the hollow element self navigates along within the body cavity following a path determined by the surrounding wall defining the body cavity.

2. A method according to claim 1 further comprising inserting an insertion head in an entrance of the body cavity for guiding the hollow element into the body cavity, wherein the insertion head has a through passageway and the hollow element progressively passes through an opening of the insertion head into the body cavity.

3. A method according to claim 1 or 2 comprising inflating the hollow element to thereby cause the hollow element to be everted and self navigate along the body cavity.

4. A method according to claim 3 further comprising monitoring pressure within the hollow element for determining whether the pressure is at or below an upper predetermined limit.

5. A method according to claim 4 further comprising regulating pressure within the hollow element during the eversion of the hollow element to maintain the pressure at or below the upper predetermined limit.

6. A method according to any one of claims 3 to 5 wherein the inflating comprises inflating the hollow element with a gas.

7. A method according to claim 6 wherein the inflating comprises pumping the gas into the hollow element to thereby cause the hollow element to be everted progressively.

8. A method according to any one of claims 3 to 5 wherein the inflating comprises introducing a liquid into the hollow element to thereby cause the hollow element to be everted progressively.

9. A method according to claim 8 wherein the hollow element is arranged within a flexible enclosure at least partly filled with the liquid, and the liquid is introduced into the hollow element to thereby cause the hollow element to be increasingly extended by forcing liquid from the enclosure into the hollow element.

10. A method according to any one of claims 1 to 9 wherein the hollow element is adapted for limiting swelling thereof during the inflation of the hollow element, for thereby preferentially causing the trailing region to be drawn along.

11. A method accordingly to claim 1 or 2 further comprising feeding the hollow element through at least one pair of counter-rotatable rolls for driving and thereby causing the hollow element to be everted progressively.

12. A method according to claim 1 wherein the body cavity is that of the uterus, urethra, ureter, bladder, oesophagus, stomach, a bronchi, a fallopian tube, a blood vessel, intestine, gastrointestinal tract, or colon.

13. A method of positioning an inflatable hollow element in a body cavity of a patient, wherein the hollow element has a leading region and a trailing region, and the method comprises:
inflating the hollow element to cause the hollow element to be everted progressively therealong from the leading region and thereby be increasingly extended into the body cavity as the trailing region of the hollow element follows along; and
wherein the hollow element is adapted for limiting swelling during the inflation thereof thereby preferentially causing the trailing region to be drawn along.

14. A method accordingly to claim 13 further comprising inserting an insertion head in an entrance of the body cavity for guiding the hollow element into the body cavity, wherein the insertion head has a through passageway and the hollow element progressively passes through an opening of the insertion head into the body cavity.

15. A method according to claim 13 or 14 further comprising monitoring pressure within the hollow element for determining whether the pressure is at or below an upper predetermined limit.

16. A method according to claim 15 further comprising regulating pressure within the hollow element during the eversion of the hollow element to maintain the pressure at or below the upper predetermined limit.

17. A method according to any one of claims 13 to 16 wherein the inflating comprises inflating the hollow element with a gas.

18. A method according to claim 17 wherein the inflating comprises pumping the gas into the hollow element to thereby cause the hollow element to be everted progressively.

19. A method according to any one of claims 13 to 16 wherein the inflating comprises introducing a liquid into the hollow element to thereby cause the hollow element to be everted progressively.

20. A method according to claim 19 wherein the hollow element is arranged within a flexible enclosure at least partly filled with the liquid, and the liquid is introduced into the hollow element to thereby cause the hollow element to be increasingly extended by forcing liquid from the enclosure into the hollow element.

21. A method of examining a patient utilising an elongate hollow element for being positioned along a body cavity of the patient, wherein the hollow element is adapted for facilitating the examination of the patient and has a leading region and a trailing region, and the method comprises:
causing the hollow element to be everted progressively along the hollow element from the leading region thereof such that the hollow element is forced to be increasingly extended and thereby progressively positioned along the body cavity; and
examining the patient.

22. A method according to claim 21 wherein a device for use in examining the patient is attached to the hollow element and is located in the body cavity by the eversion of the hollow element, and the examining comprises utilising the device to examine the patient.

23. A method according to claim 22 wherein the device is an instrument for enabling the body cavity to be viewed and the examining comprises utilising the instrument to view within the body cavity.

24. A method according to claim 22 or 23 wherein the instrument is selected from a group consisting of an endoscope, a fibroscope, a colonoscope, a bronchioscope, a laproscope, and a gastroscope.

25. A method according to claim 22 wherein the device transmits signals for enabling visualisation within the body cavity, and the examining comprises utilising an image generated from the signals to examine the patient.

26. A method according to claim 22 wherein the hollow element carriers one or more optic fibres for enabling the body cavity to be viewed and the optic fibres are located in position within the body cavity by the hollow element, and wherein the examining comprises viewing within the body cavity utilising the optic fibres.

27. A method according to any one of claims 21 to 26 further comprising withdrawing the hollow element from the body cavity, and wherein the examining comprises examining the patient as the hollow element is withdrawn from the body cavity.

28. A method of locating an instrument along a body cavity of a patient utilising an elongate hollow element, wherein the instrument is attached to the hollow element and the hollow element has a leading region and a trailing region, and the method comprises:
causing the hollow element to be everted progressively along the hollow element from the leading region thereof such that the hollow element is forced to be increasingly extended and the apparatus thereby increasingly drawn along the body cavity as the trailing region follows along, whereby the instrument is located in position within the body cavity.

29. A method accordingly to claim 28 comprising inflating the hollow element to thereby cause the hollow element to be everted and by increasingly extended.

30. A method accordingly to claim 29 wherein the hollow element is adapted for limiting swelling thereof while being inflated, for thereby preferentially causing the trailing region of the hollow element to be drawn along.

31. A method accordingly to any one of claims 28 to 30 wherein the instrument is an instrument for facilitating examination of the patient.

32. A method according to claim 31 wherein the instrument is an instrument selected from a group consisting of an endoscope, a fibroscope, a colonoscope, a bronchioscope, a laproscope, and a gastroscope.

33. A method accordingly to any one of claims 28 to 30 wherein the instrument is selected from a group consisting of an instrument for collecting a biopsy, and instrument for performing a surgical task, an instrument for cauterising a site, and an instrument for indicating a biological parameter within the body cavity.

34. A method of applying a substance to a surrounding wall defining a body cavity of a patient utilising a hollow element with an internal surface coated with the substance, wherein the hollow element has a leading region and a trailing region, and the method comprises:
causing the hollow element to be everted progressively along the hollow element from the leading region thereof such that the hollow element is forced to be increasingly extended and the interior surface of the hollow element thereby brought into contact with the surrounding wall of the body cavity as the surrounding wall is progressively lined with the hollow element and as the trailing region follows along;
wherein the hollow element self navigates along within the body cavity following a path determined by the surrounding wall defining the body cavity.

35. A method according to claim 34 comprising inflating the hollow element to thereby cause the hollow element to be everted and self navigate along the body cavity.

36. A method according to claim 34 or 35 wherein the substance is selected from a group consisting of a drug, a therapeutic compound for treating a site on the surrounding wall defining the body cavity, an agent for promoting coagulation, an isotope, and a steroid.

37. A method according to claim 36 wherein the substance is thrombin or a thrombogenic substance.

38. A method of treating a patient utilising a hollow element for being positioned along a body cavity of a patient and having a leading region and a trailing region, the method comprising:
causing the hollow element to be everted progressively along the hollow element from the leading region thereof such that the hollow element is forced to be increasingly extended and thereby progressively line a surrounding wall defining the body cavity as the trailing region follows along, to thereby effect the treatment.

39. A method according to claim 38 wherein the treating comprises utilising the hollow element to apply pressure to a site on the surrounding wall defining the body cavity.

40. A method according to claim 38 or 39 wherein the hollow element acts as a tamponade and the treating comprises treating the patient to stem blood flow.

41. A method according to claim 38 wherein the treating comprises utilising the hollow element to apply a substance to the surrounding wall.

42. A method according to claim 41 wherein the hollow element has an internal surface coated with the substance and the substance is applied to the surrounding wall defining the body cavity as the internal surface is progressively pressed against the surrounding wall as the hollow element is everted.

43. A method according to claim 42 wherein the substance is selected from a group consisting of a drug, a therapeutic compound for treating a site on the surrounding wall defining the body cavity, an agent for promoting coagulation, an isotope, and a steroid.

44. A method according to claim 43 wherein the substance is thrombin or a thrombogenic substance.

45. A method according to any one of claims 37 to 43 wherein the body cavity is that of the urethra, ureter, bladder, oesophagus, stomach, a bronchi, a fallopian tube, a blood vessel, intestine, gastrointestinal tract, or colon.

46. A device incorporating an inflatable hollow element for being positioned along a body cavity of a patient, wherein the hollow element has a leading region and a trailing region and is arranged for being everted progressively along the hollow element from the leading region thereof to thereby be increasingly extended for progressively lining a surrounding wall defining the body cavity as the trailing region follows along, and wherein the hollow element has a substantially constant diameter therealong upon being everted and fully inflated.

47. A device according to claim 46 wherein the hollow element is arranged for self navigation along the body cavity following a path determined by the surrounding wall defining the body cavity.

48. A device according to claim 46 or 47 wherein the hollow element is adapted for limiting swelling thereof during inflation of the hollow element, for thereby preferentially causing the trailing region to be drawn along.

49. A device accordingly to any one of claims 46 to 48 further comprising an enclosure in which the hollow element is located and which is provided with an opening for progressive passage therethrough of the hollow element from the interior of the enclosure, and wherein the interior of the enclosure is sealed from the opening by the hollow element.

50. A device according to claim 49 wherein the enclosure incorporates an insertion head for being inserted into an entrance of the body cavity for guiding the hollow element into the body cavity, wherein the insertion head has a through passageway defining the opening.

51. A device according to claim 50 wherein the hollow element is received in the through passageway and the leading region of the hollow element is folded back over the insertion head such that the hollow element is thereby invaginated.

52. A device according to claim 51 wherein an inlet is defined in the enclosure for the entry of a gas or liquid into the interior for progressively inflating and thereby causing to the hollow element to be progressively forced from the enclosure through the opening.

53. A device accordingly to claim 50 or 51 wherein the enclosure further comprises a flexible bag sealingly secured to the insertion head and which houses the hollow element.

54. A device accordingly to claim 53 wherein the bag is adapted for preferentially causing the hollow element to be forced from the enclosure through the opening of the insertion head upon pressure within the enclosure being increased for causing the hollow element to be inflated.

55. A device according to claim 53 or 54 wherein the bag is at least partially filled with a liquid and the device further comprises a collar receiving the bag, wherein the collar constricts the bag and is slidable along the bag toward the insertion head for forcing the liquid into the hollow element and thereby causing the hollow element to be progressively inflated.

56. A device according to any one of claims 49 to 51 further comprising a pump arranged for pumping a gas or liquid into the enclosure for increasing pressure within the enclosure to thereby cause the hollow element to be progressively everted.

57. A device accordingly to claim 56 further comprising a pressure release valve operable for enabling pressure in the interior of the bag to be decreased.

58. A device according to any one of claims 49 to 51 wherein the hollow element is arranged in a concertinaed conformation in the interior of the enclosure.

59. A device according to any one of claims 46 to 58 wherein the device is other than a catheter.

60. A device incorporating an inflatable hollow element for being positioned along a body cavity of a patient, wherein the hollow element has a leading region and a trailing region and is arranged for being everted progressively along the hollow element from the leading region to thereby be increasingly extended for achieving said positioning in the body cavity as the trailing region of the hollow element follows along, and wherein the hollow element is adapted for limiting swelling during inflation thereof for thereby preferentially causing the trailing region to be drawn along.

61. A device according to claim 60 wherein the hollow element is arranged for self navigation along the body cavity following a path determined by the surrounding wall defining the body cavity.

62. A device according to claim 60 or 61 further comprising an enclosure in which the hollow element is located and which is provided with an opening for progressive passage therethrough of the hollow element from the interior of the enclosure, and wherein the interior of the enclosure is sealed from the opening by the hollow element.

63. A device according to claim 62 wherein the enclosure incorporates an insertion head for being inserted into an entrance of the body cavity for guiding the hollow element into the body cavity, wherein the insertion head has a through passageway defining the opening.

64. A device according to claim 63 wherein the hollow element is received in the through passageway and the leading region of the hollow element is folded back over the insertion head such that the hollow element is thereby invaginated.

65. A device according to claim 64 wherein an inlet is defined in the enclosure for the entry of a gas or liquid into the interior for progressively inflating and thereby causing to the hollow element to be progressively forced from the enclosure through the opening.

66. A device according to any one of claims 62 to 64 wherein the enclosure further comprises a flexible bag sealingly secured to the insertion head and which houses the hollow element.

67. A device according to claim 66 wherein the bag is adapted for preferentially causing the hollow element to be forced from the enclosure through the opening of the insertion head upon pressure within the enclosure being increased for causing the hollow element to be inflated.

68. A device according to claim 66 or 67 wherein the bag is at least partially filled with a liquid and the device further comprises a collar receiving the bag, wherein the collar constricts the bag and is slidable along the bag toward the insertion head for forcing the liquid into the hollow element and thereby causing the hollow element to be progressively inflated.

69. A device according to any one of claims 62 to 64 further comprising a pump arranged for pumping a gas or liquid into the enclosure for increasing pressure within the enclosure to thereby cause the hollow element to be progressively everted.

70. A device according to claim 69 further comprising a pressure release valve operable for enabling pressure in the interior of the bag to be decreased.

71. A device according to any one of claims 62 to 64 wherein the hollow element is arranged in a concertinaed conformation in the interior of the enclosure.

72. A device according to any one of claim 60 to 71 wherein the device is other than a catheter.

73. Apparatus for being inserted into a body cavity of a patient and comprising:
an instrument; and
at least one inflatable hollow element having a leading region and a trailing region, and being secured to the instrument;
wherein the hollow element is arranged for being everted progressively from the leading region thereof while being inflated, to thereby be increasingly extended for progressively lining a surrounding wall defining the body cavity and drawing along the instrument as the trailing region follows along behind.

74. Apparatus according to claim 73 incorporating one said hollow element wherein the instrument extends longitudinally along a central region of the hollow element such that the hollow element thereby surrounds the instrument.

75. Apparatus according to claim 73 or 74 wherein the hollow element is adapted for limiting swelling thereof while being inflated, for thereby preferentially causing the trailing region to be drawn along.

76. Apparatus according to any one of claims 73 to 75 wherein the hollow element is arranged for self navigation along the body cavity following a path determined by a surrounding wall defining the body cavity.

77. Apparatus according to any one of claim 73 to 76 wherein the instrument is an instrument for facilitating examination of the patient.

78. Apparatus according to any one of claims 73 to 76 wherein the instrument is selected from a group consisting of an instrument for collecting a biopsy, and instrument for performing a surgical task, an instrument for cauterising a site, and an instrument for indicating a biological parameter within the body cavity.

79. Apparatus according to any one of claims 73 to 77 wherein the instrument is an instrument for facilitating visual examination within the body cavity.

80. Apparatus according to any one of claims 73 to 76 wherein the instrument is an instrument selected from a group consisting of an endoscope, a fibroscope, a colonoscope, a bronchioscope, a laproscope, and a gastroscope.

81. Apparatus according to any one of claims 73 to 76 wherein the instrument is other than a catheter.

82. Apparatus for being inserted into a body cavity of a patient and comprising:
an instrument; and
at least one inflatable hollow element secured to the instrument and for being pressed against a surrounding wall defining the body cavity;
wherein the hollow element is arranged for overlying a region of the instrument forward of a location or locations where the hollow element is secured to the instrument, for enabling the hollow element to be progressively everted upon the hollow element being inflated and the instrument being driven along within the body cavity, for thereby facilitating travel of the instrument along within the body cavity.

83. Apparatus according to claim 82 incorporating one said hollow element wherein the instrument extends longitudinally along a central region of the hollow element such that the hollow element thereby surrounds the instrument.

84. Apparatus according to claim 82 or 83 wherein the instrument incorporates one or more passageways opening into the hollow element for passage of a gas or a liquid into the hollow element for causing inflation of the hollow element.

85. Apparatus according to claim 84 further comprising one or more further passageways opening into the hollow element for subsequent passage of the gas or liquid from the hollow element for achieving deflation of the hollow element.

86. Apparatus according to any one of claims 82 to 85 wherein the instrument is for facilitating examination of the patient.

87. Apparatus according to any one of claims 82 to 86 wherein the instrument is an instrument for facilitating visual examination within the body cavity.

88. Apparatus according to any one of claims 82 to 85 wherein the instruments is selected from a group consisting of an instrument for collecting a biopsy, and instrument for performing a surgical task, an instrument for cauterising a site, and an instrument for indicating a biological parameter within the body cavity.

89. Apparatus according to any one of claims 82 to 85 wherein the instrument is an instrument selected from a group consisting of an endoscope, a fibroscope, a colonoscope, a bronchioscope, a laproscope, and a gastroscope.

90. Apparatus according to any one of claims 82 to 85 wherein the instrument is other than a catheter.

91. An arrangement incorporating an elongate hollow element for being positioned within a body cavity of a patient, comprising:
a guide head having a through passageway receiving the hollow element; and
at least one pair of opposed counter rotatable rolls for feeding the hollow element through an opening defined by the through passageway of the guide head;
wherein the hollow element has a leading region and a trailing region and is arranged for being everted progressively along the hollow element from the leading region thereof to thereby be increasingly extended from the guide head for progressively lining the body cavity upon being fed through the opening of the guide head by the counter rotatable rolls.

92. An arrangement according to claim 91 wherein the hollow element is folded back over the insertion head such that the hollow element is thereby invaginated.

93. An arrangement according to claim 91 or 92 wherein the hollow element is open at each end of the hollow element.

94. A method of locating a stent in position along a body cavity of a patient, wherein the stent has a leading region and a trailing region; and the method comprises;
causing the stent to be everted progressively along the stent from the leading region thereof such that the stent is forced to be increasingly extended and thereby progressively line a surrounding wall defining the body cavity as the trailing region follows along.

95. A method according to claim 94 wherein the stent overlays an inflatable hollow element arranged in an invaginated conformation, and wherein the method further comprises inflating the hollow element to thereby cause the stent to be progressively everted as the hollow element is everted.

96. A method according to claim 95 wherein the inflating of the hollow element forces the stent to radially expand within the body cavity and be pressed against the surrounding wall.

97. A stent for being positioned along a body cavity of a patient, wherein the stent has a leading region and a trailing region and is arranged for being everted progressively therealong from the leading region to thereby be increasingly extended for progressively lining a surrounding wall defining the body cavity as the trailing region follows along.

98. A device for use in treating a patient and comprising:
an inflatable hollow element; and
a stent having a leading region and a trailing region and which is arranged for being everted progressively therealong from the leading region to thereby be increasingly extended for progressively lining a surrounding wall defining a body cavity as the trailing region follows along;
wherein the stent overlays the hollow element and the hollow element is arranged in an invaginated conformation for causing the stent to be everted for thereby lining the surrounding wall of the body cavity upon the hollow element being inflated.
